(19) United States
(12) Patent Application Publication
(10) Pub. No.: US 2006/0258965 A1
(43) Pub. Date: Nov. 16, 2006

(54) Dynamic supinated splint

(76) Inventors: Michael J. Lee, Tucson, AZ (US); Ann E. vonKersburg, Tucson, AZ (US); Paul C. Lastayo, Salt Lake City, UT (US)

Correspondence Address: CHRISTIE, PARKER & HALE, LLP, PO BOX 7068, PASADENA, CA, 91109-7068, US

(21) Appl. No.: 10/549,864

(22) PCT Filed: Mar. 24, 2004
(86) PCT No.: PCT/US04/09069

§ 371 Date: May 23, 2006

Related U.S. Application Data
(60) Provisional application No. 60/457,177, filed on Mar. 24, 2003.

(51) Int. Cl. A61F 5/00

(52) U.S. Cl. 602/20; 602/5

(57) ABSTRACT

A dynamic supinated splint for increasing supination of a subject is described having a splint body with a distal hand support section and a proximal forearm support section. The hand and forearm of a subject are strapped to the splint body using a plurality of straps. A supinated force is generated using a force generator anchored to the splint body through hooking arrangements and an outrigger in a diagonal arrangement for creating a torque or a moment.

Splints for increasing passive and active range of motions for forearm supination are generally discussed herein with particular discussions extended to below elbow dynamic supinated splints for added elbow flexion and extension functionality.

### BACKGROUND

Forearm rotation is necessary for various daily activities, such as, e.g., feeding, dressing, and performing functions related to personal hygiene. It is also an integral component of motion for many vocations and avocations. Normal forearm rotation is approximately 0° to 80° or 90° for both supination and pronation. A functional arc of forearm rotation is 100° (50° of supination and 50° of pronation). While the loss of pronation may be compensated for by shoulder abduction, no degree, or at least no significant degree, of shoulder or elbow compensation, can restore function when there is a significant loss of forearm supination.

Forearm supination is dependent upon the complex interplay between, among other things, the distal radioulnar joint (DRUJ), interosseous membrane, and the proximal radioulnar joint (PRUJ). Injuries or pathologies affecting any of these areas can potentially lead to loss of forearm supination (and/or pronation). Common conditions include: distal radius fractures, radial head fractures, Galeazzi and Monteggia fractures, Essex-Lopresti injury and any surgical procedures which change any of the structures listed above.

Various prior art dynamic splints have been designed to assist in increasing supination, with one of the first splints reported in 1944. In this earlier splint, the elbow is fixed at 90°, the wrist and hand are splinted in neutral, rubber bands are attached from a forearm piece to the radial and ulnar sides of the hand/wrist piece to create rotation. Although this early splint is no longer formally used, it has served as a template for more current forearm rotation splints.

Presently, two of the more frequently used dynamic forearm rotation splints, both of which cross the flexion/extension joints of the elbow, are the Colello-Abraham dynamic pronation/supination splint and a commercially available dynamic supination/pronation kit made available by Smith and Nephew Rolyan, Inc., Germantown, Wis. The Collelo-Abraham splint consists of a humeral cuff, two lateral bars running parallel to the forearm, and a cock-up splint with multiple rings, to which rubber bands are attached from the lateral bars to provide the rotational force. One of the advantages of the Colello-Abraham splint is that the use of multiple force arms increases the area of force application and thus, decreases pressure and improves comfort. The commercial splint kit employs a twisted rubber tube to generate the rotational force. One of the advantages of this splint is that it may be more time efficient, as construction of an outrigger is not required.

A significant drawback with the dynamic forearm rotation splints used to date is that the elbow is fixed at 90°. While this elbow flexed position at 90° optimizes the attachment site for components to be located proximally on the aforementioned splints, the lack of elbow motion with currently available splints can limit the patient's functional use (i.e. eating, drinking, grooming, etc) of the splinted extremity, as the elbow is fixed at a 900 angle and does not permit any flexion or extension. Hence, this drawback often leads to decrease patient wear time of the splints. Decrease wear time negatively impacts treatment as it is well known in the art that the longer a splint is worn, the greater the total end range time (TERT), and the greater the return in passive range of motion (PROM).

Accordingly, there is a need for a splint that dynamically supinates the forearm but does not cross the elbow flexion and extension joints and hence, does not fix the elbow in flexion. This configuration allows the wearer adequate flexion and extension of the elbow for activities related to daily living (ADLs). In addition, there is a need for a splint that allows the patient to temporarily rotate the forearm from supination to pronation to perform ADLs, as necessary. Clinically, it is apparent that if function can be maintained, it is more likely the patient will wear the splint for longer periods of time. Still yet, there is a need for a splint that is less time consuming to construct and less costly to produce than the prior art splints.

### SUMMARY

The present invention may be implemented by providing a dynamic supinated splint comprising a splint body comprising an axis having a first strap for fixing a first part of an arm to the splint body and a second strap for fixing a second part of the arm to the splint body; the splint body further comprising first anchor and a second anchor and an outrigger comprising two generally vertical sections and a generally horizontal section disposed in between the first anchor and the second anchor and having an end of each of its vertical sections secured to the splint body such that the outrigger transects the axis of the splint body; wherein a force generator is engaged to the first anchor and the second anchor at its two ends and is expanded at a point in between its two ends by the horizontal section of the outrigger to provide a torque to the splint body.

In another aspect of the present invention, there is provided a dynamic supinated splint comprising a splint body comprising a proximal end, a distal end and an axial shaft; a hand support section on the distal end comprising two folded flaps configured to cover a first metacarpal and a fifth metacarpal, or at least a portion thereof, when worn by a subject; a forearm support section comprising a curved section extending laterally of the axial shaft, the curved section terminating just distal of a lateral epicondyle and partially covering at least a portion of a radius and ulna of a forearm when the splint is worn by the subject; and a force generator comprising two ends mechanically coupled to the splint body for generating a torque to the splint body.

In yet another aspect of the present invention, there is provided a dynamic supinated splint comprising a longitudinal splint body comprising a central shaft made from a pliable splint material, the splint body comprising a distal hand support section comprising two flaps rolled inwardly toward the central axis of the longitudinal splint body, an opening at the distal hand support section having an area forming part of one of the two flaps; an undulating section on part of the longitudinal splint body; and a proximal forearm support section comprising a curved section having a portion arced laterally from the longitudinal splint body; wherein a distal anchor and a proximal anchor are coupled to the splint body and a force generator comprising two ends coupled to the two anchors to provide a force to create a bending moment on the longitudinal splint body.

### BRIEF DESCRIPTION OF THE DRAWINGS

These and other features and advantages of the present invention will become appreciated as the same become better understood with reference to the specification, claims and appended drawings wherein:

FIG. 1 is semi-schematic side view of a below elbow dynamic supinated splint provided in accordance with aspects of the present invention;

FIG. 2 is a semi-schematic plan view of a plurality of splint components usable in making the splint of FIG. 2;

FIG. 3 is a semi-schematic view of the splint of FIG. 1 from a different angle;

FIG. 4 is a semi-schematic view of the splint of FIG. 1 from another angle;

FIG. 5 is a semi-schematic view of the splint of FIG. 1 from yet another angle; and

FIG. 6 is a semi-schematic view of the splint of FIG. 1 worn by a subject.

### DETAILED DESCRIPTION

The detailed description set forth below in connection with the appended drawings is intended as a description of the presently preferred embodiments of a below elbow dynamic supinated splint provided in accordance with practice of the present invention and is not intended to represent the only forms in which the present invention may be constructed or utilized. The description sets forth the features and the steps for constructing and using the splint of the present invention in connection with the illustrated embodiments. It is to be understood, however, that the same or equivalent functions and structures may be accomplished by different embodiments that are also intended to be encompassed within the spirit and scope of the invention. Also, as denoted elsewhere herein, like element numbers are intended to indicate like or similar elements or features.

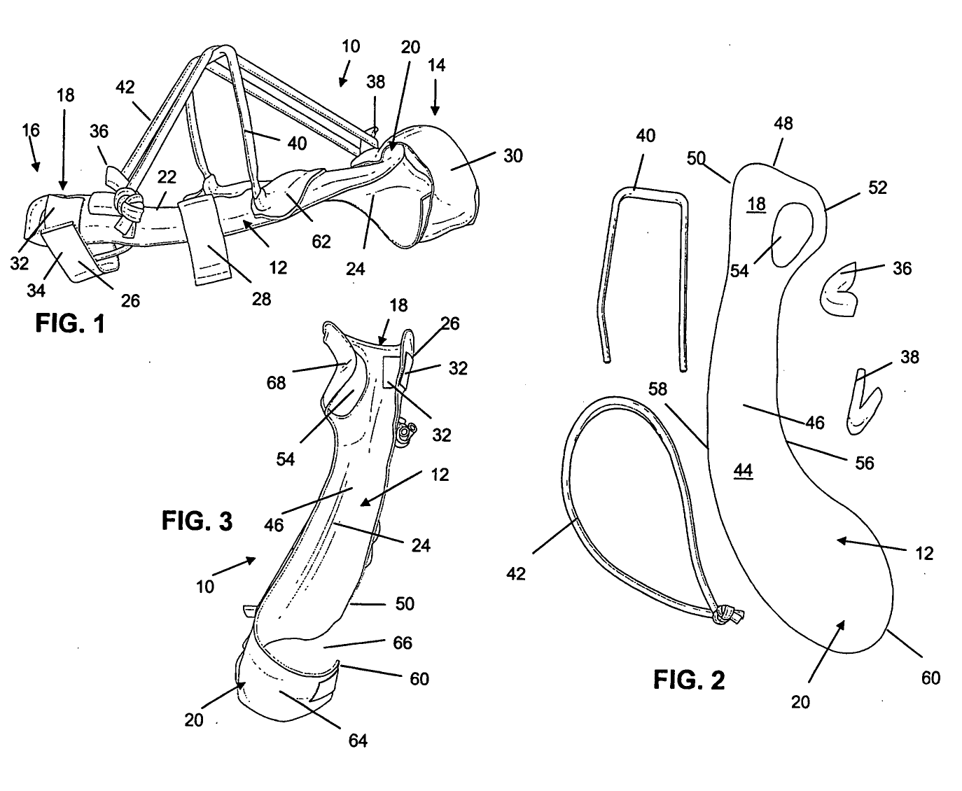

Referring now to FIG. 1, there is shown a below elbow dynamic supinated splint (“splint”) provided in accordance with aspects of the present invention, which is generally designated 10. In an exemplary embodiment, the splint 10 comprises a splint body 12 comprising a proximal end 14 and a distal end 16. A hand and wrist support section or distal splint base 18 is located at the distal end 16 of the splint body 10 while a forearm support section or proximal splint base 20 is located at the proximal end 14.

The splint body 12 comprises an exterior surface 22 and an interior surface 24, which defines a contact surface for contacting the palmar side of the hand, wrist, and anterior, radial, posterior, and ulnar of the forearm section, as further discussed below. Exteriorly, the splint body comprises a plurality of straps, which in one embodiment, includes a distal strap 26, a middle strap 28, and a proximal strap 30. The plurality of straps can be of the Velcro® type or equivalent. In the present embodiment, each strap location comprises a hook 32 and a loop 34 strap component. Preferably, the hook component comprises an adhesive backing.

Also exteriorly, the splint 10 comprises means for generating a dynamic force for assisting in increasing supination of the forearm of the wearer of the splint, herein the subject. In one exemplary embodiment, the means comprises a set of anchors 36, 38, an outrigger 40, and a resilient or elastic force generator 42, which may comprise a coiled spring, a rubber band, or a rubber tube, such as a Theratube®. If a coiled spring is used, the anchors 36, 38 and the outrigger 40 may be modified accordingly to facilitate gripping the two ends of the coiled spring and supporting a center section of the coiled spring. As further discussed below, the splint body and the means for generating a force are adapted to increase the subject's passive range of motion.

Referring now to FIG. 2, the splint components are shown in a pre-assembled state. In the pre-assembled state, the splint body 12 is first patterned 44 as shown. The pattern 44 includes a palm/wrist support section 18, a forearm support section 20, and a shaft section 46. The splint body 12 may be made by trimming the pattern 44 from any number of prior art splinting materials, such as original Aquaplast®, Aquaplast®-T, Aquaplast® Watercolors, solid or perforated, and any variety of available thicknesses, including 3/16″, ⅛″, 3/32/″, and 1/16″ with ⅛″ being more preferred. The splint material is a polymer based material with polycaprolactone being a preferred polymer. Once formed, the pattern 44 may be used for making a left-handed splint or, by turning the pattern 180 degrees or upside down, a right-handed splint. As discussed herein, the pattern 44 is to be placed on the palmar side of a hand, wrist, and anterior surface of the forearm section of a subject to make a right-handed splint. As readily apparent, the pattern 44 can be pre-made or pre-cut in several standard sizes for a large built individual, a medium built, a small built, a child, etc. with final trimming to be performed on site when forming the splint for a particular subject. Alternatively, the pattern 44 can be tailored cut or trimmed from a raw sheet of splinting material when fitting a subject.

Referring initially to the palm support section 18, the pattern 44 is first made by trimming a sheet of splint material to produce a distal edge 48 and two sides 50, 52. This palm support section 18 should be wider than the width of a palm so that the first side 50 can fold around the fifth metacarpal and the second side 52 can fold around the first metacarpal. The distal edge 48 should lie just proximal of the base of the fingers or distal palmar crease. However, in a preferred embodiment, the distal edge 48 is to be folded backwards or proximally to just proximal of the base of the fingers during the forming step to eliminate sharp edges. An opening 54 is provided between the two sides 50, 52 for the thumb or first metacarpal access. The opening 54 resembles a water drop but may embody any number of shapes. Generally speaking, the palm support section 18 should be sized sufficiently to allow thumb CMC mobility and full metacarpal phalangeal joint flexion.

The shaft section 46 extends proximally of the palm support section 18 approximately one-third of the length of the radius then arcs laterally. Thus, the shaft section 46 should comprise at least one interior curved section or radius 56 and one exterior curved section or radius 58. The distal shaft section, which comprises the forearm support section 20, should lie laterally closer to the first metacarpal side of the edge 52 than the fifth metacarpal side of the edge 50. The terminal end 60 of the forearm support section 20 should have a curved or a smooth contour. As further discussed below, the pattern 44 is then formed on an arm and cured to resemble the splint body 12 in FIG. 1.

Also shown in FIG. 2 are the anchors 36, 38, the outrigger 40, and the force generator 42. In one exemplary embodiment, the anchors 36, 38 may each be made by folding a 3 inch×2 inch rectangular patch of splint material and bending the patch at approximately its center position to form a “V” shape. The anchors 36, 38 can also be strengthened or assisted in maintaining the “V” shape before curing by incorporating a metal rod or an insert in the center of the patch. Alternatively, metal hooks may be used in making the anchors without the splint patch material.

The outrigger 40 may be constructed by placing a 1/16-inch copper wire inside of a ⅛-inch Aquatube® for providing structure to the molding material before heating and settling. The outrigger 40, before bending into the U-shaped configuration shown, is about 13-inches in length. When shaped, the outrigger 40 is approximately 3-inches wide and 5-inches high. The effective height, however, is only about 4½ inches high as about ½-inch of each leg is bent for attaching the bent portions of the outrigger to the splint body 12, as further discussed below. However, the dimensions of the outrigger can vary depending on the desired torque or force to be generated by the force generator 42. As readily apparent, for a given force generator 42, the higher or taller the outrigger 40 (i.e., different dimension), the more torque may be generated by the force generator. The ½-inch bent ends on the two vertical sections may be attached to the splint body 12 using two square patches 62 (FIG. 1) of splinting material, one on each end of the outrigger. Alternatively, a metal rod or tubing may be used to form the outrigger without the Aquatube®.

In one exemplary embodiment, the force generator 42 is made by folding a length of a rubber tube Theratube® and tying a knot at the loose end. The length of the rubber tube 42 may be variable depending upon the length of each subject's forearm, the subject's tolerance to the induced force, and degree of stiffness of the splint body. However, once mounted onto the two hooks 36, 38 and prior to placing the Theratube® over the outrigger 40 (FIG. 1), the tied rubber tube should not have any slack.

Referring now to FIG. 3, the interior surface 24 of the splint body 12 is shown with the forearm support section 20 curved or arced laterally around an axis define by the forearm of a subject, i.e., around the radius and ulna of the forearm. This curved section 64 of the forearm support section 20 together with the shaft section 46 should extend about ¾ to about ⅞ of the circumference of the forearm just distal of the lateral epicondyle, at the area of the radial head. This arrangement leaves an open gap 66 between the first side 50 of the splint body and the terminal end 60 of the curved section for mounting and dismounting the splint 10 onto a forearm.

Also shown in FIG. 3 is an interior hook 32 of the distal strap 26 and an exterior hook 32. In one exemplary embodiment, the distal strap 26 is used to strap in a palm by attaching one end of a loop 34 (not shown) section of the strap to the interior hook 32, running the free end of the loop 34 section through the opening 54 and around the bridge section 68 of the opening, then attaching the free end of the loop 34 section to the exterior hook 32.

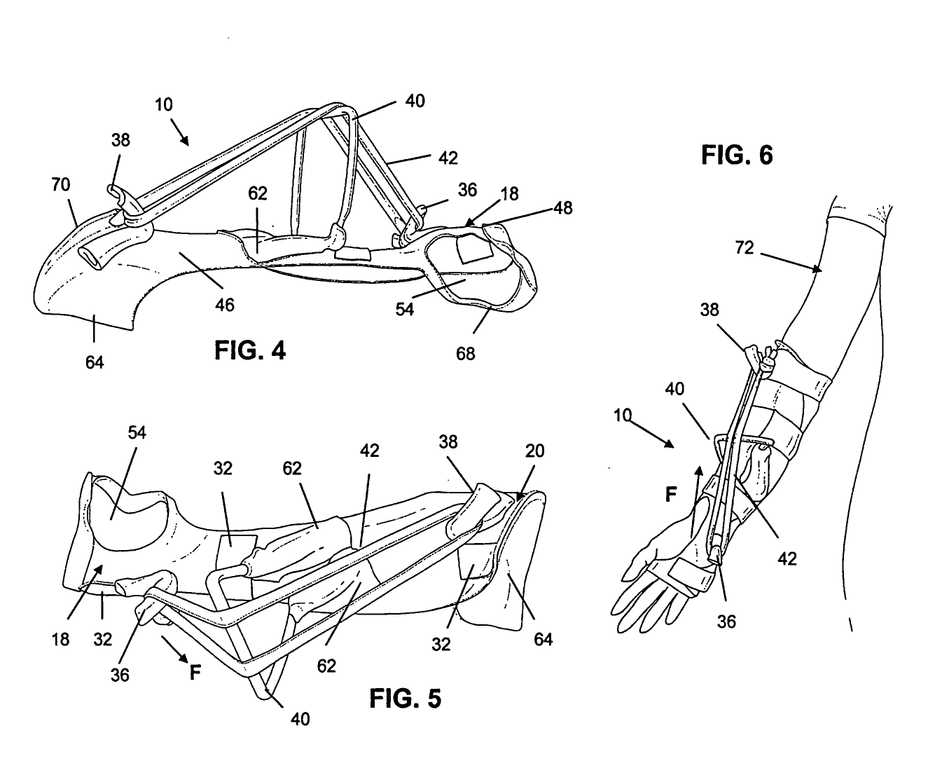

FIG. 4 is a reverse view of the splint 10 of FIG. 1 without the loop traps 34 at the three strap locations for clarity. As clearly shown in the exemplary embodiment of FIG. 4, the distal edge 48 at the palm support section 18 has been folded proximally and cured in the folded position to eliminate sharp edges. Similarly, an end portion 70 of the shaft section 46 near the curved section 64 has also been folded radially away from the forearm to eliminate sharp edges.

FIG. 5 is another semi-schematic view of the splint of FIG. 1 also without the loop traps 34 at the three strap locations for clarity. In the view shown, the two legs of the outrigger 40 are bent and directed or pointed proximally. However, it is possible to turn the bent portions distally. Also shown in FIG. 5 is the direction of the dynamic force F generated by the force generator 42. The force F, as further discussed below, provides a dynamic supinated force F for assisting in increasing supination of the forearm of a subject.

FIG. 6 is semi-schematic view of the splint 10 mounted on or worn by a subject 72. The splint 10 is shown with four strap locations, with fewer or more straps being acceptable. When worn, the splint 10 provides a dynamic force tangential to the elastic tubes of the force generator 42 to assist in increasing supination of the forearm of the subject 72. The force generator 42 connects to points at both the distal ulnar wrist level and the proximal radial forearm and lies over the rotational axis of the forearm. Thus, a torque is generated by the force generator at both the distal and proximal ends of the splint body 12. The distal force generated at the distal end creates a supination moment while the proximal force generated at the proximal end creates an equal and opposite pronation moment. The torque or moment at both ends is calculated by multiplying the force generated by the force generator 42 by a perpendicular distance of that force from the axis of rotation of the forearm.

However, only a supination torque is desired. Therefore, the proximal force generated at the proximal end, which is anchored by the proximal anchor 38, should be minimized or eliminated. Traditionally, the proximal force is cancelled by placing the proximal attachment above the elbow so that the humerus can effectively cancel the pronation moment. However, this option fixes the elbow at a 90 degree angle and inhibits functional elbow motion while the subject wears the splint 10. In the presently preferred embodiment, the proximal torque is eliminated by the curved section 64 of the splint body 12 wrapping posteriorly from the lateral forearm to near the medial epicondyle. This configuration eliminates the pronation moment and does so without necessarily inhibiting functional elbow motion.

The basis premise of the splint 10 is a “corkscrew” about the axis of forearm rotation which biases the forearm toward a supinated position. The splint 10 may be formed from the components shown in FIGS. 1-6, and particularly in FIG. 2, by first applying the pattern 44 on a subject in a forearm based neutral wrist splint position. The pattern is molded or manipulated around the subject by squeezing and pressing the splinting material to the palm and then progressing proximally. At approximately one-third of the radius length, the pattern arcs laterally around the radius. The pattern 44 progresses circumferentially around the forearm, ending slightly medial and distal to the medial epicondyle.

When fitting the splint body 12 or pattern, the subject should be placed in a supine position, shoulder flexed to approximately 45 degrees, and elbow extended. The pattern 44 is placed volarly, as in fitting a basic splint. As discussed above, at the palm support section 18, adequate room must be provided to allow thumb CMC mobility and full metacarpal phalangeal joint flexion.

After the distal end is secured, the splint body 12 is wrapped radially and dorsally. The radial side of the splint body 12 should extend to just distal of the lateral epicondyle, at the area of the radial head. The splint body 12 continues to wrap circumferentially around the forearm, concluding slightly distal and medial to the medial epicondyle.

The outrigger 40 is next placed on the splint body 12. The outrigger 40 is placed at approximately the mid-radius section at an angle so that it transects the long axis of the forearm, which is approximately a line from the radial head to the ulnar styloid. The outrigger 40 may be secured to the splint body 12 using patches 62 of Aquaplast®.

To provide a base for the force generator 42, two hooks 36, 38, made from 3 inch×2 inch patches of Aquaplast®, are secured to the splint body 12. In one exemplary embodiment, the hooks 36, 38 are placed at: (1) the ulnocarpal joint distally and (2) the radial head proximally. The hooks should be positioned along an imaginary line corresponding to the axis of the forearm rotation. For this reason, the outrigger 40 and the hooks 36, 38 should be placed on the splint while the subject is wearing the splint.

A securing strap 26 is then placed dorsally at the metacarpals to secure the wrist and hand. Another strap 28 is placed approximately mid-forearm to secure the forearm in position. Finally a strap 30 is placed proximally to span from the ulnar end of the splint to the lateral/radial side of the forearm. Optionally, a fourth strap may be used between the distal strap 26 and the middle strap 28 (FIG. 6).

A force generator 42, such as a rubber tube from Theratube® is then tied, as to form a loop, with each end attached to a hook 36, 38. Once the ends are secured, the force generator 42 is lifted over the outrigger 40 to provide the dynamic tension.

The effectiveness of the splint 10 provided in accordance with aspects of the present invention is discussed below in a retrospective evaluation of a study conducted using eleven patients, 2 males and 9 females, from 1998-2000. The subjects had various elbow and wrist fractures which led to a loss of forearm supination (TABLE 1).

TABLE 1

| Subject | Diagnosis | Fixation |
|---|---|---|
| 1 | Distal Radius Fracture | ORIF |
| 2 | Distal Radius and Ulna Fracture | |
| | with Ulnar Osteotomy | |
| 3 | Distal Radius Fracture | Cast |
| 4 | Distal Radius Fracture | External Fixator |
| 5 | Distal Radius Fracture | External Fixator |
| 6 | Distal Radius Fracture and | ORIF |
| | Osteotomy | |
| 7 | Radial Head Fracture/Excision | Cast |
| 8 | Proximal Ulna and Trochlea | ORIF |
| | Fracture | |
| 9 | Radial Head Fracture/Excision | None |
| 10 | Distal Radius Fracture | Cast |
| 11 | Distal Radius Fracture | ORIF |

The subjects' ages ranged from 38-70 years, with an average of 48.3 years. All patients were right hand dominant with almost an equal distribution of injuries to the dominant or non-dominant extremity. Patients were seen for treatment ranging from 5 to 26 visits, with an average of 17.7 visits, over an average of 10.0 weeks (Table 2). The dynamic supination splint was, on average, issued on the fifth visit.

TABLE 2

| Sex: | | |
|---|---|---|
| Male | 2 | subjects (18%) |
| Female | 9 | subjects (82%) |
| Age: | | |
| Range | 38-70 | years |
| Average | 48.3 | years |
| Hand Dominance: | | |
| Right | 11 | subjects (100%) |
| Left | 0 | subjects (0%) |
| Involved Hand: | | |
| Right | 6 | subjects (55%) |
| Left | 5 | subjects (45%) |

All treatments, which addressed loss of forearm supination, were identical for all subjects both before and after splint application. Treatments consisted of: passive range of motion (PROM), active-assistive range of motion (AAROM), active range of motion (AROM), soft tissue mobilization to the pronators, resistive forearm rotation exercises, and moist heat while placed in a supination stretch utilizing a weight. The decision to splint was made either due to: 1) inadequate range of motion (ROM) gains or 2) per physician request due to limited ROM. An inadequate ROM gain was defined as the point when improvements in supination ROM became recalcitrant to the above-described treatment techniques. Subjects were instructed to wear the dynamic supination splint at least 4 total hours per day, progressing to a maximum of 8 total hours. Duration of wearing time per wearing session and the number of times the splint was worn per day was determined by patient tolerance, with the total daily hours within the 4-8 hour limit.

Goniometric measurements for PROM (TABLE 3) and AROM (TABLE 4) were taken after preconditioning, i.e. the restricting soft tissues had achieved their maximum length (without causing damage) via cyclic loading.

TABLE 3

| | No. of Visits | | | | | | | | | | | | | | | | | | | |
|---|---|---|---|---|---|---|---|---|---|---|---|---|---|---|---|---|---|---|---|---|
| Subject: | 1 | 2 | 3 | 4 | 5 | 6 | 7 | 8 | 9 | 10 | 11 | 12 | 13 | 15 | 18 | 19 | 20 | 23 | 25 | 26 |
| 1 | 0 | + | | 50 | | | | | | 80* | | | | | | | | | | 90 |
| 2 | + | | | | 70 | | 70 | | | | | | 80* | | | | | | 80 | |
| 3 | 50 | + | | 80* | | | | | 90 | | | | | | | | | | | |
| 4 | 55+ | 90* | | | 90 | | | | | | | | | | | | | | | |
| 5 | 60 | | | + | | | 75* | | | | 80 | | | | 80 | | | | | |
| 6 | 55+ | 80* | 80 | | | | | | | | | | | | | | | | | |
| 7 | 70 | | + | | | | | 80* | | | | | 90 | | | | | | | |
| 8 | 0 | | | | | | | | | | | | + | 60* | | 65 | 65 | | | |
| 9 | 20 | | | | | | + | | 50 | | | 60* | | | | 70 | | 80 | | |
| 10 | 0 | | | | 10 | 20 | | | | + | | 40 | 50* | | 60 | | | | | 80 |
| 11 | 40 | | | | + | 45 | 50 | 55* | | 60 | | 70 | | 75 | 80 | | | | | |

Blank spaces indicate that no ROM measurements were obtained on that date. Due to the retrospective nature of this review, measurements were not obtained at regular intervals other than on the initial and final visit.
*Indicates measurement used for middle phase of rehabilitation, which was determined as the closest visit on which a ROM measurement was made to the total number of visits divided by two
+ Indicates visit when a splint was applied, which was determined by inadequate ROM gains or per physician referral.

TABLE 4

| | Visit Number | | | | | | | | | | | | | | | | | | |
|---|---|---|---|---|---|---|---|---|---|---|---|---|---|---|---|---|---|---|---|
| Subject | 1 | 2 | 3 | 4 | 5 | 7 | 8 | 9 | 10 | 11 | 12 | 13 | 15 | 18 | 19 | 20 | 23 | 25 | 26 |
| 1 | 0 | + | | | | | | | 70* | | | | | | | | | | 80 |
| 2 | + | | | | 50 | 50 | | | | | | 50* | | | | | | 70 | |
| 3 | 50 | + | 70* | | | | | 90 | | | | | | | | | | | |
| 4 | 50+ | 70* | | | 80 | | | | | | | | | | | | | | |
| 5 | 50 | | | + | | 70* | | | | 70 | | | | 80 | | | | | |
| 6 | 50+ | 60* | 70 | | | | | | | | | | | | | | | | |
| 7 | 40 | | + | | | | 60* | | | | | 70 | | | | | | | |
| 8 | 0 | | | | | | | | | | | + | 45* | | 50 | 55 | | | |
| 9 | 30 | | | | | + | | | | | 50* | | | | | | 80 | | |
| 10 | 0 | | | | | | | | + | | | 40* | | | | | | | 50 |
| 11 | 0 | | | | + | | 45* | | | | | | 60 | 70 | | | | | |

Blank spaces indicate that no ROM measurements were obtained on that date. Due to the retrospective nature of this review, measurements were not obtained at regular intervals other than on the initial and final visit.
*Indicates measurement used for middle phase of rehabilitation, which was determined as the closest visit on which a ROM measurement was made to the total number of visits divided by two
+ Indicates visit when splint was applied, which was determined by inadequate ROM gains or per physician referral.

Goniometric measurements were taken as described by Norkin and White, Measurement of Joint Motion: A guide to Goniometry, 2nd ed. Philadelphia, Pa., 1995. The subjects were positioned in sitting with the shoulder in 0° of flexion, extension, abduction, adduction, and rotation so that the upper arm was next to the side of the body. The elbow was then flexed to 90° and the center of the goniometer was positioned lateral to the ulnar styloid process. One arm of the goniometer was aligned with the anterior mid-line of the humerus and the other was placed across the volar aspect of the forearm, just proximal to the styloid processes.

A repeated measure analysis of variance (ANOVA) was utilized to determine statistical significance between subjects and between phases of rehabilitation. Phases of rehabilitation were defined as: initial, middle, and discharge. As measurements were not originally taken at specific intervals, the middle phase of rehabilitation value was determined as the closest measurement to the total number of visits divided by two. A post-hoc Tukey multiple pair wise comparison test was also utilized to isolate differences between each phase of rehabilitation.

For the radiographic analysis, one female subject, who was not a subject in the retrospective review as she had no previous history of injury or ROM limitations, was positioned for a standard wrist variance film. The shoulder was abducted to 90°, the elbow was flexed to 90°, and the film was then taken posterior to anterior. Three films in the wrist variance position were taken for analysis: resting position without the splint, maximal active forearm supination without the splint, and passive position of the forearm in supination while wearing the splint. An additional radiograph was taken with the shoulder abducted to 90°, elbow fully extended, and humerus internally rotated. The forearm was passively supinated by the splint and the radiograph was taken posterior to anterior as with the wrist variance view. The radiographs are reproduced below:

[image]

A radiographic and electromyographic (EMG) analysis was performed using the same subject as the radiographic analysis. Bipolar surface, silver chloride electrodes, with an inter-electrode spacing of 2 cm and a detector surface diameter of 1 cm, were utilized. One electrode was placed over the supinator muscle and a second was placed over the bicep. A common ground was placed over the ipsilateral scapula. Multiple trials were performed to differentiate wrist extensor versus supinator muscle activity to determine optimal electrode placement. EMG analysis was used to study three conditions: 1) resting, quiescent muscle position, 2) maximal isometric supination contraction, and 3) resting passively in a supinated position while in the splint. EMG signals were pre-amplified, digitized at 500 Hz, and later analyzed on a computer. During the analysis, the raw data was rectified and peak activity was averaged for each of the three conditions.

A repeated measure ANOVA was utilized to determine statistical significance between the EMG measurement conditions. A post-hoc Tukey multiple comparison test was then utilized to further specify significant differences between the measurement conditions.

Subjects showed improvements with use of the splint 10 provided in accordance with aspects of the present invention. Average PROM increased from the initial rehabilitation phase to middle phase and also from the middle to discharge phases of rehabilitation (TABLE. 5).

[image]

The greatest increase was from an initial average of 34.0° to an average of 71.8° at the middle phase of rehabilitation. PROM then increased from an average of 71.8° at the middle phase of rehabilitation to 82.3° at discharge. Significant differences in average PROM between subjects and between phases of rehabilitation (p<0.001) were noted. Post-hoc analysis revealed significant average PROM differences between initial and middle phases (p<0.05), but not between middle and discharge phases of rehabilitation.

Average AROM also increased from initial to middle and from middle to discharge phases of rehabilitation. The greatest increase was from an initial average of 27.0° to an average of 57.3° at the middle phase of rehabilitation. AROM then increased from an average of 57.3° at the middle phase of rehabilitation to 72.3° at discharge. ANOVA results demonstrated statistically significant differences in average AROM between subjects and between phases of rehabilitation (p<0.001). Post-hoc Tukey testing also revealed statistically significant average AROM differences between initial and middle phases and middle and discharge phases of rehabilitation (p<0.05).

The X-ray film images indicate that the radius and ulna alignment is nearly identical between active supination and the passive, resting supinated position in the dynamic supination splint regardless of elbow position. EMG results determined average supinator muscle activity as follows: 7.8 mV (SE=0.0004) at rest, 7.8 mV (SE=0.0004) when splinted in supination, and 68.0 mV (SE=0.004) during a maximal isometric effort (FIG. 6). Relative supinator muscle activity, when splinted in supination, was found to be 98.7% of the average resting value and 11.5% of the maximal effort EMG.

ANOVA results indicate a statistically significant difference between the three EMG measurement conditions (p<0.001). Post-hoc Tukey testing determined a statistically significant difference between the resting and splinted EMG values versus the maximal effort EMG value (p<0.05), but there was no significant difference between resting versus splinted EMG values.

The multiple patient cases indicate that AROM and PROM increased significantly from the beginning to the end of therapy. PROM increased to an average of 82.3°, which falls within the normal range of 80°-90°. AROM, however, did not fall within this range with an average of 72.3°. This is not unexpected as the dynamic splint is a passive modality and AROM should improve with weaning from the splint and increased strengthening and functional use. It is possible that the increase in AROM is likely more a result of the other active treatments (AAROM, AROM, and resistive exercise) rather than the passive dynamic supination splint. The only change in ROM that was not statistically significant was the increase in PROM from middle to discharge phases of rehabilitation. This may indicate that the greatest benefit from the splint was obtained during the initial to middle phases of rehabilitation when large gains in ROM were possible. Therefore, the conclusion that the dynamic supination splint assisted significantly in increasing PROM can be made, at least as it applies to the subjects within this descriptive study.

The EMG data clearly indicates that the splint is a passive modality. It was previously believed that the dynamic supination splint must have a proximal attachment, above the elbow, to generate an adequate passive supination force. “Adequate” is defined as a force significant enough to place the forearm in a supinated position. The combination of the radiographic images and the EMG data indicate that the splint does passively position the forearm in supination, despite the fact that the proximal margin does not cross the elbow.

These assessment approaches were used so as to couple the clinical outcomes with some information on the mechanical effectiveness of the splint. Heretofore, no studies exist that provide clinical outcome data utilizing dynamic supination splinting. Therefore, a comparison of outcomes versus alternative treatment/splinting techniques was not conducted, nor is it possible to generalize the use of this splint with other patients beyond these described here. However, it is believed that the retrospective increase in supination ROM, coupled with the radiographic images and EMG data make a compelling argument as to the merits of this splint.

Experience suggests that the less functionally inhibiting a splint, the more often the patient will wear the splint. It has been reported that the longer a splint is worn, greater total end range time (TERT), the greater the return in PROM. As stated previously, all other dynamic supination splints cross the elbow, thus requiring the elbow to be fixed at 90° and inhibiting functional elbow motion while wearing the splint. The supination splint 10 provided in accordance with aspects of the present invention does not cross the elbow, thereby allowing functional elbow flexion and extension. Since the splint 10 is dynamic, the patient may also temporarily pronate the forearm as needed for function. This dual ability to flex and extend the elbow and temporarily pronate the forearm will increase the patient's functional use and thus, should increase compliance and splint wearing time.

Other important factors in patient compliance are comfort and ease of donning/doffing. Subjectively, no subjects reported limiting their wearing time due to discomfort. All subjects also demonstrated the ability to don and doff the splint independently. This is important as patients that live alone must be able to manage the splint with one hand.

Although limited embodiments of the dynamic splint have been specifically described and illustrated herein, many modifications and variations will be apparent to those skilled in the art. Accordingly, it is to be understood that the splint and its components constructed according to principles of this invention may be embodied other than as specifically described herein. The invention is defined in the following claims.

What is claimed is:

1. A dynamic supinated splint comprising a splint body comprising an axis having a first strap for fixing a first part of an arm to the splint body and a second strap for fixing a second part of the arm to the splint body; the splint body further comprising first anchor and a second anchor and an outrigger comprising two generally vertical sections and a generally horizontal section disposed in between the first anchor and the second anchor and having an end of each of its vertical sections secured to the splint body such that the outrigger transects the axis of the splint body; wherein a force generator is engaged to the first anchor and the second anchor at its two ends and is expanded at a point in between its two ends by the horizontal section of the outrigger to provide a torque to the splint body.

2. The dynamic supinated splint as recited in claim 1, where the horizontal section of the outrigger comprises a length that is less than a length of either vertical section of the outrigger.

3. The dynamic supinated splint as recited in claim 2, wherein the end of each vertical section is formed from bending a portion of each vertical section.

4. The dynamic supinated splint as recited in claim 1, wherein the splint body comprises a proximal section, the proximal section comprising a curved section extending laterally the axis of the splint body.

5. The dynamic supinated splint as recited in claim 4, wherein the curved section terminating just distal of a lateral epicondyle and partially covering at least a portion of a radius and ulna of a forearm when the splint is worn by a subject.

6. The dynamic supinated splint as recited in claim 5, wherein the second strap connects the curved section with the axis portion of the splint body.

7. The dynamic supinated splint as recited in claim 6, further comprising a third strap disposed in between the first strap and the second strap.

8. The dynamic supinated splint as recited in claim 6, wherein the two straps each comprises a hook strap component and a loop strap component.

9. The dynamic supinated splint as recited in claim 7, wherein the three straps each comprises a hook strap component and a loop strap component.

10. The dynamic supinated splint as recited in claim 1, wherein the splint body comprises a distal hand support section comprising two folded flaps configured to cover a first metacarpal and a fifth metacarpal, or at least a portion thereof, when the splint is worn by a subject.

11. The dynamic supinated splint as recited in claim 10, wherein the distal hand support portion further comprises an opening having an area forming part of one of the two folded flaps.

12. The dynamic supinated splint as recited in claim 1, wherein the force generator comprises a rubber tube.

13. The dynamic supinated splint as recited in claim 12, wherein the rubber tube is bent at approximately a center section and tied at a loose end of the rubber tube to form a loop.

14. The dynamic supinated splint as recited in claim 13, wherein the tied end and the bent section are attached to the first and second anchors.

15. The dynamic supinated splint as recited in claim 1, wherein the splint body is made from a polymer.

16. The dynamic supinated splint as recited in claim 15, wherein the polymer is a polycaprolactone base made from Aquaplast®.

17. The dynamic supinated splint as recited in claim 16, wherein the splint body is made from a ⅛″ thick sheet of Aquaplast® splint material.

18. The dynamic supinated splint as recited in claim 1, wherein the two straps comprise straps made from Velcro®.

19. The dynamic supinated splint as recited in claim 1, further comprising a distal hand support section, said distal hand support section comprising a distal end bend in a proximal direction for eliminating sharp edges.

20. A dynamic supinated splint comprising a splint body comprising a proximal end, a distal end and an axial shaft; a hand support section on the distal end comprising two folded flaps configured to cover a first metacarpal and a fifth metacarpal, or at least a portion thereof, when worn by a subject; a forearm support section comprising a curved section extending laterally of the axial shaft, the curved section terminating just distal of a lateral epicondyle and partially covering at least a portion of a radius and ulna of a forearm when the splint is worn by the subject; and a force generator comprising two ends mechanically coupled to the splint body for generating a torque to the splint body.

21. The dynamic supinated splint as recited in claim 20, wherein the force generator is mechanically coupled to two anchors, which are secured to the splint body.

22. The dynamic supinated splint as recited in claim 20, wherein the force generator is made from a rubber tube.

23. The dynamic supinated splint as recited in claim 22, wherein the rubber tube is bent at approximately a center section and tied at a loose end of the rubber tube to form a loop.

24. The dynamic supinated splint as recited in claim 23, wherein the tied end and the bent section are attached to the two anchors.

25. The dynamic supinated splint as recited in claim 20, wherein the splint body is made from a polymer.

26. The dynamic supinated splint as recited in claim 25, wherein the polymer is a polycaprolactone base made from Aquaplast®.

27. The dynamic supinated splint as recited in claim 26, wherein the splint body is made from a ⅛″ thick sheet of Aquaplast® splint material.

28. The dynamic supinated splint as recited in claim 20, further comprising an outrigger, the outrigger comprising a generally horizontal section and two generally vertical sections.

29. The dynamic supinated splint as recited in claim 28, wherein the two generally vertical sections are attached at a base of each vertical section to the splint body.

30. The dynamic supinated splint as recited in claim 29, wherein each attached base comprises a bent section of each generally vertical section.

31. The dynamic supinated splint as recited in claim 30, wherein the attached base is each attached to the splint body by a patch of splint material.

32. The dynamic supinated splint as recited in claim 20, further comprising a plurality of straps connected to the splint body, each strap comprising a hook component and a loop component.

33. A dynamic supinated splint comprising a longitudinal splint body comprising a central shaft made from a pliable splint material, the splint body comprising a distal hand support section comprising two flaps rolled inwardly toward the central axis of the longitudinal splint body, an opening at the distal hand support section having an area forming part of one of the two flaps; an undulating section on part of the longitudinal splint body; and a proximal forearm support section comprising a curved section having a portion arced laterally from the longitudinal splint body; wherein a distal anchor and a proximal anchor are coupled to the splint body and a force generator comprising two ends coupled to the two anchors to provide a force to create a bending moment on the longitudinal splint body.

34. The dynamic supinated splint as recited in claim 33, wherein the pliable splint material comprises one of Aquaplast®, Aquaplast®-T, and Aquaplast® Watercolors.

35. The dynamic supinated splint as recited in claim 33, wherein the force generator comprises a rubber tube.

36. The dynamic supinated splint as recited in claim 33, wherein the distal anchor and the proximal anchor are each made from rolling a patch of splint material into a V-shape body.
